(19) United States
(12) Patent Application Publication
(10) Pub. No.: US 2021/0236580 A1
(43) Pub. Date: Aug. 5, 2021

(54) Methods and Compositions for Mitigating Symptoms of Acute Respiratory Distress Syndrome

(71) Applicant: Sytheon Ltd, Boonton, NJ (US)

(72) Inventors: Ratan K Chaudhuri, Lincoln Park, NJ (US); Manpreet Randhawa, Robbinsville, NJ (US)

(73) Assignee: Sytheon Ltd, Boonton, NJ (US)

(21) Appl. No.: 17/035,405

(22) Filed: Sep. 28, 2020

Related U.S. Application Data
(60) Provisional application No. 63/006,501, filed on Apr. 7, 2020.

(51) Int. Cl. A61K 36/87; A61K 36/185; A61K 36/53; A61K 36/36; A61K 36/81; A61K 31/573

(52) U.S. Cl. CPC A61K 36/87; A61K 36/185; A61K 31/573; A61K 36/36; A61K 36/81; A61K 36/53

(57) ABSTRACT

A method of treating or inhibiting the symptoms of acute respiratory distress syndrome, especially that associated with Covid-19, said method comprising administering an effective amount of a cytokine and/or innate immune response modulating natural extract, its components or metabolites, or combinations thereof capable of modulating those cytokine and/or innate immune response associated mechanisms associated with acute respiratory distress.

### RELATED APPLICATIONS

The present application claims the benefit of prior U.S. Provisional Patent Application No. 63/006,501, filed Apr. 7, 2020, entitled “Methods and Compositions for Mitigating Symptoms of Acute Respiratory Distress Syndrome,” the contents of which are hereby incorporated herein by reference in their entirety.

### FIELD OF THE INVENTION

The present disclosure relates to the use of select cytokine modulating natural extracts, their components and/or metabolites, for mitigating symptoms of acute respiratory distress syndrome and/or for enhancing the immune response, particularly the initial immune response, of T cells and/or NK cells associated with a number of diseases and microbial infections, especially viral infections such as those associated with various influenza and coronaviruses, in humans. In particular, the present teaching is directed to a method of mitigating or preventing the manifestation or occurrence of cytokine storms and/or enhancing the production of interferon gamma in individuals infected with an influenza and/or a coronavirus.

### BACKGROUND OF THE INVENTION

Prior research has suggested that high levels of IL-6 and IL-8—two, key biomarkers for inflammation and a high-level immune response—is associated with a higher mortality rate in people with community-acquired pneumonia. Severe acute respiratory syndrome (SARS), which is caused by the SARS coronavirus (SARS-CoV), is a highly communicable disease with the lungs as the major pathological target. Although SARS likely stems from overexuberant host inflammatory responses, the exact mechanism leading to the detrimental outcome in patients remains unknown. Pulmonary macrophages (Mφ), airway epithelium, and dendritic cells (DC) are key cellular elements of the host innate defenses against respiratory infections. While pulmonary Mφ are situated at the luminal epithelial surface, DC reside abundantly underneath the epithelium. Such strategic locations of these cells within the airways make it relevant to investigate their likely impact on SARS pathogenesis subsequent to their interaction with infected lung epithelial cells, In the lead-up to the present discovery, a study was conducted to investigate this using highly polarized human lung epithelial Calu-3 cells by using the Transwell culture system. It was found that supernatants harvested from the apical and basolateral domains of infected Calu-3 cells are potent in modulating the intrinsic functions of Mφ and DC, respectively. They prompted the production of cytokines by both Mφ and DC and selectively induced CD40 and CD86 expression only on DC. However, they compromised the abilities of the DC and Mφ in priming naïve T cells and phagocytosis, respectively. Oher researchers have also identified interleukin-6 (IL-6) and IL-8 as key SARS-CoV-induced epithelial cytokines capable of inhibiting the T-cell-priming ability of DC (T Yoshikawa, et al., Severe Acute Respiratory Syndrome (SARS) Coronavirus-Induced Lung Epithelial Cytokines Exacerbate SARS Pathogenesis by Modulating Intrinsic Functions of Monocyte-Derived Macrophages and Dendritic Cells, J Virology, 83(7):3039-3048, 2009). Taken together, these results provide insights into the molecular and cellular bases of the host antiviral innate immunity within the lungs that eventually lead to an exacerbated inflammatory cascades and severe tissue damage in SARS patients.

When a coronavirus infects a cell, it dumps its genetic payload—a single strand of RNA containing the recipes for making proteins it needs to replicate—into its host. The immune system mobilizes to kill infected cells before too many copies of the virus can be made. Sometimes, however, that defense mechanism overreacts whereby healthy cells, as well as the sick cells, are killed and a lot of them. Fortunately, most patients do develop their own response against the virus and recover from it, but some patients just have a very brisk response and get really sick.

The lungs constitute a portal of entry for various respiratory pathogens, and, fortunately, evolution has equipped this vital organ with elaborate host defense systems to maintain its sterility and normal respiratory functions. Epithelium, pulmonary M, and dendritic cells (DC) are three key cellular elements of the airway innate immune system. In addition to functioning as physical and mechanical barriers that separate and eliminate many inhaled materials, lung epithelial cells can directly respond to respiratory infection by secreting various molecules to initiate and sustain cascades of inflammatory responses that ultimately influence the development of adaptive immune responses required to sterilize the infection (L D Martin et al., Airway epithelium as an effector of inflammation: molecular regulation of secondary mediators. Eur. Respir. J. 10:2139-2146, 1997; A J Polito et al., J. Allergy Clin. Immunol. 102:714-718, 1998). Although this early epithelial response is beneficial in facilitating pathogen clearance, an unregulated and excessive epithelial response can also lead to exacerbated inflammatory responses, causing severe tissue damage (J M Stark et al., Respiratory syncytial virus infection enhances neutrophil and eosinophil adhesion to cultured respiratory epithelial cells. Roles of CD18 and intercellular adhesion molecule-1. J. Immunol. 156:4774-4782, 1996).

Viral infections, especially those association with influenza and coronavirus, are often widespread, global in nature with varying mortalities. For example, in the 2019-2020 flu season in the US, influenza has manifested a mortality rate of 0.095%; yet, the novel coronavirus, now identified as SARS-CoV-2, which is the cause of COVID-19 and the source of the current ongoing pandemic, is already showing at least a 2% mortality rate worldwide, with much higher levels in certain regions. It remains to be seen what the true number will be on a national as well as a world-wide basis, but it is uncontested that influenza will pale in comparison to the wrath of SARS-CoV-2. Critically important studies emerging from China (Q Ruan et al., Clinical predictors of mortality due to COVID-19 based on an analysis of data of 150 patients from Wuhan, China, intensive Care Med, https://doi.org/10.1007/s00134-020-05991-x. 2020) suggest that for many patients who die of Covid-19, it may be their own immune system, rather than the virus itself, that deals the fatal blow as a result of a cytokine storm.

During a cytokine storm, an excessive immune response ravages healthy lung tissue, leading to acute respiratory distress and multi-organ failure. Untreated, cytokine storm syndrome is usually fatal. Patients in other studies (G S Schulert et al., Whole-Exome Sequencing Reveals Mutations in Genes Linked to Hemophagocytic Lymphohistiocytosis and Macrophage Activation Syndrome in Fatal Cases of H1N1 Influenza, J Infect Dis, 213(7):1180-1188, 2016) who developed cytokine storm syndrome after viral triggers often ironically are subsequently found to have possessed subtle genetic immune defects resulting in the uncontrolled immune response.

One common cause of cytokine storms is the over-expression of interleukin-6 (IL-6), one of the most important pro-inflammatory cytokines and one which has been involved in a wide range of disease occurrence and pathogenesis. In two gene therapy clinical trials, the surge of IL-6 was attributed to the cytokine storm and related adverse effects. (T Bian et al., Over-expression of Interleukin-6 alone induces dexamethasone-relieved multiple-organ lesion in mice, Immunologic & Host responses in Gene & Cell Therapy, Vol 21, Supplement 1, S173, May 1, 2013, DOI:https://doi.org?10.1016/S1525-0016(16)34884-0). In an animal study, T Bien et al demonstrated that the acute phase symptoms induced by AAV-IL-6 (recombinant adeno-associated virus (AAV) vector expressing murine IL-6) were partially prevented and the organ damages were alleviated by Dexamethasone. Bone lesions were dramatically recovered and serum paraproteins were largely eliminated. Overall, the results showed that IL-6 alone could potently induce multiple organ inflammatory response, suggesting that IL-6 plays a critical role during the pathological process.

The aforementioned Ruan et al., study revealed that there was a significant difference in age between the death group and the discharge group (p<0.001) but no difference in the sex ratio (p=0.43). A total of 63% (43/68) of patients in the death group and 41% (34/82) in the discharge group had underlying diseases (p=0.0069). It should be noted that patients with cardiovascular diseases had a significantly increased risk of death when they are infected with SARS-CoV-2 (p<0.001). A total of 16% (11/68) of the patients in the death group had secondary infections, and 1% (1/82) of the patients in the discharge group had secondary infections (p=0.0018). Laboratory results showed that there were significant differences in white blood cell counts, absolute, values of lymphocytes, platelets, albumin, total bilirubin, blood urea nitrogen, blood creatinine, myoglobin, cardiac troponin, C-reactive protein (CRP) and interleukin-6 (IL-6) between the two groups.

In association with the current Covid-19 pandemic, doctors in China noted that in some sick patients, viral levels dropped, but levels of IL-6—one of the distress signals used to call the immune system to action—remained high. A small study was conducted to test whether Actemra (tocilizumab), a humanized anti-IL-6R monoclonal antibody, would be effective in modulating or interfering with progression of the symptoms of Covid-19. Preliminary findings from a single-arm, 21-patient Chinese trial found that the Covid-19 patients experienced rapidly reduced fevers, with 75% of patients experiencing a reduced need for supplemental oxygen, after treatment with Actemra.

In addition to the issues with inflammation as discussed above, another factor associated with pathogens, especially bacteria, viruses and the like, and the human response to the same is the extent to which the interferon response is induced. Specifically, it is known that many antagonists that should elicit an immune response, fail to do so or do so very poorly. The interferon response is critical for providing an efficient protection against various pathogens, especially bacterial and viral pathogens. While most pathogens illicit or induce an immediate interferon response which initiates a cascade of processes leading to the attack and control of the pathogens, certain pathogens are found to be poor inducers of the interferon response: this is particularly true for certain viruses, especially the coronaviruses such as SARS-Cov-2. Indeed, the lack of or ineffective interferon response seems to be a hallmark of many coronaviruses. In following, it seems that coronaviruses have developed multiple strategies to escape and counteract innate sensing and interferon production. Consequently, the delayed interferon response promotes the accumulation of pathogenic monocyte-macrophages and enhances disease severity. Additionally, the problem is exacerbated in immune compromised and elderly individuals who naturally have reduced levels of the key interferons, especially interferon gamma. It is believed that this has, at least in part, contributed to the higher susceptibility to and mortality by SARS-CoV-2.

The present pandemic has once again shown the world that it is not ready to deal with the myriad of unknown and/or yet to form viruses. Despite past instances of Avian flu, SARS as well as the annual influenza viruses, there are still no effective treatments to mitigate the acute respiratory distress syndrome associated with advance cases. Furthermore, the increasing happenstance of cytokine storms indicate that simply seeking treatments to stop, kill or, at least, slow down the replication and progression of the virus is not sufficient. Rather, efforts must also be directed to addressing and controlling the immunological processes of the patients themselves. Accordingly, the medical community must first be aware of the possibility, then make the diagnosis, and finally treat infected individuals with overly active immune responses that are harmful, if not fatal, if left untreated.

Consequently, there is need not only to address acute respiratory distress syndrome, there is also a need to induce and/or enhance the interferon response, particularly that associated with interferon alpha (IFN-α) and interferon gamma (IFN-γ).

### SUMMARY

According to a first aspect of the present teaching there is provided a method for treating acute respiratory distress syndrome, most especially for preventing and/or mitigating the manifestation of acute respiratory distress syndrome, particularly that associated with a number of diseases and microbial infections, said method comprising administering to an individual manifesting inflammation of the respiratory system or suffering from a disease or infection, particularly a bacterial or viral infection, that induces inflammation of the respiratory system an effective amount of select cytokine modulating natural extracts, their components and/or metabolites which natural extracts down regulate those cytokines responsible for inflammation of the respiratory system, most especially those cytokines that induce and/or enhance hyperinflammation of the respiratory system and/or cytokine storms. Most preferably the cytokine modulating natural extracts, their components and/or metabolites are those that are capable of down regulating interleukin 6 (IL-6) and/or interleukin 8 (IL-8) and/or their corresponding downstream genes or the production thereof.

According to a second aspect of the present teaching there is provided a method for inducing, promoting and/or enhancing the immune response of T cells, NKT cells and/or NK cells upon exposure to or following exposure to various pathogenic microorganisms, especially viruses, said method comprising administering to individuals exposed to and/or infected with said pathogenic microorganisms, especially bacteria and viruses, most especially viruses, an effective amount of select cytokine and/or innate immune response modulating natural extracts, their components and/or metabolites which natural extracts up regulate those cytokines responsible for promoting and/or enhancing the immune response of T cells, NKT cells and/or NK cells. In particular, the method is especially useful in inducing or promoting the immune response to those exposures and/or infections which are known or found, to fail to induce or poorly induce the interferon response, particularly the interferon alpha and interferon gamma responses, most especially the interferon gamma response: particularly those infections associated with various influenza viruses and coronaviruses, most especially the Sars viruses including, in particular, SARS-CoV-2, which is the cause of COVID-19. Most preferably the cytokine and/or innate immune response modulating natural extracts, their components and/or metabolites are those that are capable of upregulating interleukin 12 (IL-12) and/or interferon gamma (IFN-γ), and/or their corresponding downstream genes or the production thereof.

The cytokine and/or innate immune response modulating natural extracts, their components and/or metabolites include, by way example, but not limitation, almond extract, Occimum gratissmium, Occimum sanctum, Mollugo pentaphylla L, Hypericum triquetrifolium, Ampelopsis brevipedunculata (Maxim.) Trautv. (AB), Withania somnifera root, Terminalia chebula fruits, Terminalia bellerica fruits, Terminalia arjuna, Emblica officinalis fruits, especially hydrolysable tannin-rich plant extract(s) and/or terpenes, such as tannin-rich Terminalia chebula fruit extracts, and combinations thereof. The cytokine and/or innate immune response modulating natural extracts, their components and/or metabolites, may be used alone or in combination with antimicrobial agents, especially antiviral agents (e.g., remdesevir, hydroxychloroquine, etc.), and/or with other therapeutic agents such as plasma treatments, antibody treatments (e.g., Tocilizumab), and the like. The combination treatment is believed synergistic in helping patients recover from acute respiratory distress syndrome, especially from that associated with influenza and coronavirus infections.

Finally, the present teaching is also directed to the aforementioned cytokine and/or innate immune response modulating natural extracts, their components and/or metabolites, for use in the treatment of acute respiratory distress syndrome, most especially in preventing and/or mitigating the manifestation of acute respiratory distress syndrome, associated with a number of diseases and microbial infections and/or in inducing, promoting and/or enhancing the immune response of T cells, NKT cells and/or NK cells, most especially in inducing or increasing the production of interleukin 12 (IL-12) and/or interferon gamma (IFN-γ), upon exposure to or following exposure to a pathogenic microorganism, most especially a virus.

### DETAILED DESCRIPTION

For purposes of better understanding the present teachings to be appreciated that the following terms have the meanings presented.

“Preventing” or “prevention” refers to reduce the risk of manifesting the named symptom or condition, especially acute respiratory distress syndrome.

The concept of “treating” refers to the act of reversing, alleviating, arresting, inhibiting, mitigating or ameliorating at least one of the clinical symptoms associated with acute respiratory distress syndrome, inhibiting the progression of acute respiratory distress syndrome, as well as delaying the onset of at least one or more symptoms of acute respiratory distress syndrome in a patient who has been exposed to or is infected with a microbe, especially a viral agent, that induces or is associated with the manifestation of acute respiratory distress syndrome. In following, treating also refers to inhibiting acute respiratory distress syndrome, either physically, (e.g., stabilization of a discernible symptom), physiologically, (e.g., stabilization of a physical parameter), or both, and to inhibiting at least one physical parameter that may or may not be discernible to the patient.

“Improve” or “improvement” is used to convey the fact that the cytokine modulating natural extract, its component(s) and/or metabolite(s), has manifested or effected changes, most notably beneficial changes, in either the characteristics and/or the physical attributes of the tissue to which it is being provided, applied or administered, including, for example, reduced inflammation, reduced IL-6 and/or IL-8, etc., and/or an enhanced immunological response, particularly with respect to an inducement or increase in IL-12, IFN-alpha and/or IFN-gamma. These terms are also used to indicate that the symptoms or physical characteristics associated with the diseased state are diminished, reduced or eliminated.

“Inhibiting” generally refers to delaying the onset of the symptoms, delaying or stopping the progression of the symptoms, alleviating the symptoms, or eliminating the symptoms associated with acute respiratory distress syndrome.

“Inducing” or “enhancing” is used to convey the sense that an upregulation occurs with respect to and/or in the production of the named response, feature or component. For example, the induction or enhancement of IL-12, IFN-α and/or IFN-γ means that their levels and/or those of their corresponding downstream genes are higher following administration of the select natural extracts, their components and/or metabolites, as compared to their levels prior to the administration and/or in the absence thereof.

“Rich” when used in combination with tannin or terpene means that the natural extract or component or metabolite thereof has a high tannin and/or terpene contract, typically at least 20% by weight, preferably at least 30% by weight, most preferably 40% by weight or more.

According to a first aspect of the present teaching there is, provided a method for treating acute respiratory distress syndrome, most especially preventing and/or mitigating the manifestation of acute respiratory distress syndrome, particularly that arising from or associated with a number of diseases and microbial infections, said method comprising administering to an individual manifesting inflammation of the respiratory system or suffering from a disease or infection, particularly a bacterial or viral infection, that induces inflammation of the respiratory system an effective amount of select cytokine modulating natural extracts, their components and/or metabolites, which natural extracts down regulate those cytokines responsible for inflammation of the respiratory system, most especially those cytokines that induce and/or enhance hyperinflammation of the respiratory system and/or cytokine storms. In particular, the method involves the administration of cytokine modulating natural extracts, their components and/or metabolites, capable of down regulating interleukin 6 (IL-6) and/or interleukin 8 (IL-8) and/or their corresponding downstream genes or the production thereof.

Although the present method is employed with individuals suffering respiratory inflammation from any of a number of sources, including environmental exposures, e.g., chemical exposure, smoke, etc., the present method is most especially directed to the treatment of individuals who have been exposed to and/or are manifesting symptoms associated with infection by a pathogenic microorganism, especially an influenza virus or a coronavirus, most especially SARS-CoV and SARS-CoV-2: the latter the cause of Covid-19. Furthermore, while the present method is employed with individuals already manifesting some degree of respiratory distress syndrome, it may also be used as a prophylactic treatment designed and used to prevent respiratory distress syndrome disease from occurring, particularly where continued production of inflammatory cytokines, especially IL-6 and/or IL-8, is excessive and/or continues despite lessening of other symptoms associated with the disease.

According to a second aspect of the present teaching there is provided a method for inducing, promoting and/or enhancing the immune response of T cells, NKT cells and/or NK cells upon exposure to or following exposure to various pathogenic microorganisms, especially viruses, said method comprising administering to individuals exposed to and/or infected with said microbial species, especially bacteria and viruses, most especially viruses, an effective amount of select cytokine modulating natural extracts, their components and/or metabolites which natural extracts are capable of up regulating those cytokines responsible for inducing, promoting and/or enhancing the immune response of T cells, NKT cells and/or NK cells. In particular, the method is especially useful in inducing or promoting the immune response to those infections or irritants which are known or found to fail to induce or poorly induce the interferon response, particularly the interferon alpha and interferon gamma responses, most especially the interferon gamma response. As with the preceding method, the present method may be used in association with any agent that effects an immune response such as irritants and pathogenic microorganisms; however, it is especially beneficial for use in addressing exposures and/or infections associated with various pathogenic microorganisms, especially influenza viruses and coronaviruses, most especially the SARS viruses including, in particular, SARS-CoV-2, which is the cause of COVID-19. In particular, this method involves the administration of cytokine modulating natural extracts, their components and/or metabolites, capable of upregulating interleukin 12 (IL-12), interferon alpha, and/or interferon gamma (IFN-γ) and/or their corresponding downstream genes or the production thereof, most especially interleukin 12 and/or interferon gamma.

The critical element of the methods presented herein is the select cytokine and/or innate immune response modulating natural extracts, their components and/or metabolites, in particular those extracts, components and/or metabolites that are capable of down regulating IL-6 and/or IL-8 and/or up regulating IL-12, interferon alpha and/or interferon gamma, most especially IL-12 and interferon gamma, and/or the corresponding downstream genes thereof. For convenience and expediency, these extracts, components and metabolites are hereinafter collectively and individually referred to as the “cytokine modulating agents.” Suitable cytokine modulating agents include, by way example, but not limitation, almond extract, Occimum gratissmium, Occimum sanctum, Mollugo pentaphylla L, Hypericum triquetrifolium, Ampelopsis brevipedunculata (Maxim.) Trautv. (AB), Withania somnifera root, Terminalia chebula fruits, Terminalia bellerica fruits, Terminalia arjuna, Emblica officinalis fruits, and the like, their components and/or metabolites, as well as combinations of the foregoing. Especially beneficial are the hydrolysable tannin-rich plant extract(s) and/or terpenes, such as tannin-rich Terminalia chebula fruit extracts, and combinations thereof, most especially tannin rich Terminalia chebula fruit extract, tannin rich Emblica officinalis fruit extract or tannin rich Terminalia bellerica fruit extract or tannin rich Terminalia arjuna. Additional extracts can readily be identified by simple gene assay evaluation employing human bronchial epithelial cells and/or ciliated airway tissues and looking at their effect on the key interleukins and interferons noted herein.

As noted, each method involves the administration of an effective amount of the cytokine modulating agents: an effective amount being evidenced by a manifestation of an improvement, inhibition, and/or benefit with respect to the purpose for which the cytokine modulating agent is being applied. In particular, administration of an effective amount of the cytokine modulating agent(s) will prevent, delay, inhibit and/or improve the manifestation of hyperinflammation of the respiratory system and/or cytokine storms and/or induce, enhance and/or more quickly initiate an immune response, particularly in the respiratory system. Preferably, the methods involve the administration of an amount of one or more cytokine modulating agents which effect at least a 20% down regulation in IL-6 and/or IL-8 and/or their corresponding downstream cytokine/chemokine and/or at least a 20% up regulation in IL-12, IFN-alpha and/or IFN-gamma and/or their corresponding downstream cytokine/chemokine as opposed to the response to the same trigger in the absence of the cytokine modulating agent: down regulation and up regulation being evidenced by a reduction or inhibition or a promotion or enhancement, respectively, in the expression or generation/production of the aforementioned interleukins and/or interferons and/or their corresponding downstream cytokine/chemokine, as appropriate. More preferably, the extent of the modulation, i.e., the down regulation and/or up regulation, is at least a 30%, most preferably at least a 50%, as compared to the same trigger in the absence of the cytokine modulating agent.

The cytokine modulating agents may be used individually or in combination. They may also be used in combination with antimicrobial agents, especially antiviral agents (e.g., remdesevir, hydroxychloroquine, etc.), and/or with other therapeutic agents such as plasma treatments, antibody treatments (e.g., Tocilizumab), and the like, known or subsequently identified to address and/or treat the cause of the disease or condition being addressed.

The specific amount of the cytokine modulating agents to be administered to a given patient will vary depending upon the specific cytokine modulating agent used, the delivery method, the specific disease and/or trigger for the event being addressed (e.g., chemical exposure, bacterial infection, viral infection, etc.), the weight of the patient, etc. The comparative efficacy of the various agents, as well as combinations thereof, can be ascertained by simple trial and error and/or by in-vitro assessment of gene expression. Administration of the cytokine modulating agents enable patients to recover faster from acute respiratory distress and/or other manifestations of the immune response being addressed, reduce or lessen the severity of the acute respiratory distress and/or other manifestations of the immune response, and reduce the risk of death from acute respiratory distress, especially from that associated with influenza and coronavirus infections, most especially COVID-19. Additionally, while the administration of the cytokine modulating agent is preferably performed upon manifestation of the immune response to the trigger, most especially, when indications are of acute respiratory distress such as hyperinflammation and/or cytokine storm, it may also be performed as a preventative measure, prior to exposure to the trigger, especially the microbe or virus, or at least prior to the manifestation of adverse symptoms, or at least prior to the manifestation of acute respiratory distress. The latter is particularly pertinent if other symptoms of the disease or exposure are subsiding while the inflammatory cytokines, especially IL-6 and/or IL-8 continue to rise.

As noted, the cytokine modulating agent may be administered as a preventative prior to exposure to the pathogen or trigger or, preferably, is administered in advance of the manifestation of the symptoms associated with the infection, e.g., following a known exposure, but before diagnostic confirmation: especially in the case of those triggers or pathogenic microorganisms that are known to have a low or poor induction of the immune response. More preferably, the cytokine modulating agent is administered following manifestation of the symptoms of the disease and/or infection or trigger, especially upon manifestation of inflammation of the respiratory system, most especially upon the incidence of hyperinflammation and/or cytokine storm. Notwithstanding the foregoing, because of the key roles played by IL-6 and IL-8 in the immune-response system, it is important not to administer the cytokine modulating agent or too much thereof too early in the course of the infection or immune response whereby it interferes with the nature response to the infection or invasion as this may lead to an earlier and faster progression of the disease. On the other hand, it is preferable to have initiated administration of the treatment once adverse respiratory symptoms are manifesting, most especially, once other symptoms of the infection, disease or other trigger are starting to decrease or wane, e.g., if fever is dropping, achiness is less severe, etc. Most preferably, the administration of the cytokine modulating agent will have begun if the level of IL-6 and/or IL-8 continues to rise or stays at elevated levels even though other symptoms of the infection or disease or another trigger appear to be subsiding. In this respect, it is especially desirable to have initiated the treatment prior to the commencement of hyperinflammation and/or a cytokine storm.

The cytokine modulating agents in a proper delivery vehicle may be used alone or in combination with various antimicrobial agents, especially antibiotics and/or antiviral agents, and/or with other therapeutic agents such as plasma treatments, antibody treatments (e.g., Tocilizumab), and the like and/or in combination with other anti-inflammatory agents, antioxidants, vitamins and the like. Indeed, it is believed that the aforementioned combinations are not only cumulative in their benefits but provide synergy in helping patients with enhanced immune response and/or to lessen the manifestation of and/or recover from acute respiratory distress syndrome, especially from that associated with influenza and coronavirus infections. Selection will depend, in part, upon the particular trigger, infection or microbe being addressed. For example, indications are that azithromycin, hydroxychloroquine, chloroquine, and combinations thereof may be effective in the treatment of Covid-19. Hence, their combination with the cytokine modulating agents of the present teaching are believed beneficial, if not synergistic.

The cytokine modulating agents of the present teaching are preferably administered as a composition comprising the cytokine modulating agent and a pharmaceutically acceptable vehicle such as a pharmaceutically acceptable diluent, a pharmaceutically acceptable adjuvant, a pharmaceutically acceptable excipient, a pharmaceutically acceptable carrier, or a combination of any of the foregoing. Such vehicles are well known and standard in the pharmacological art. Exemplary carriers include fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like. Finally, again as noted, these compositions may further comprise other pharmacological active agents which do not destroy, have a marked adverse effect on or interfere with the activity of the therein contained cytokine modulating agent.

The physical form of the pharmaceutical compositions containing the cytokine modulating agent for use in the present method may vary depending upon a number of factors such as the intended target, the nature of the cytokine modulating agent itself, the mode of administration, and the like.

Solid form preparations include powders, tablets, pills, capsules, cachets, suppositories, lozenges, and dispersible granules. A solid carrier can be one or more substances which may also act as diluents, flavoring agents, solubilizers, lubricants, suspending agents, binders, preservatives, tablet disintegrating agents, or an encapsulating material including, for example, magnesium carbonate, magnesium state, talc, sugar, lactose, pectin, dextrin, starch, gelatin, tragacanth, chewing gum, methylcellulose, sodium carboxy-methlycellulose, a low melting wax, cocoa butter, and the like. In powders, the carrier is a finely divided solid, which is in a mixture with the finely divided active component. In tablets, the cytokine modulating agent is mixed with the carrier having the necessary binding capacity in suitable proportions and compacted in the shape and size desired.

Liquid preparations include solutions, suspensions, and emulsions, for example, water or water-propylene glycol solutions. For example, parenteral injection liquid preparations can be formulated as solutions in aqueous polyethylene glycol solution. The cytokine modulating agents may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the cytokine modulating agent may be in powder form, obtained by aseptic isolation of sterile solid or by lyophilization from solution, for constitution with a suitable vehicle, e.g. sterile, pyrogen-free water, before use.

Aqueous solutions suitable for oral or inhalation use can be prepared by dissolving or suspending the cytokine modulating agent in water and adding suitable colorants, flavors, stabilizing and thickening agents, as desired. Alternatively, depending upon the cytokine modulating agent and its physical properties, aqueous suspensions suitable for oral use can be made by dispersing the finely divided cytokine modulating agent in water with viscous material, such as natural or synthetic gums, resins, methylcellulose, sodium carboxy-methylcellulose, or other well-known suspending agents. Compositions suitable for oral administration in the mouth includes lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in suitable liquid carrier.

Finally, solutions or suspensions may be applied directly to the nasal cavity by conventional means, for example with a dropper, pipette, or spray. Similarly, solutions or suspensions may be applied directly to the respiratory tract by conventional means, for example, by a spray, nebulizer, or inhaler. The compositions may be provided in single or multi-dose form. In compositions intended for administration to the respiratory tract, including intranasal compositions. The suspension or solutions or active will generally have a small particle size for example of the order of 5 microns or less. Such a particle size may be obtained by means known in the art, for example by micronization, atomization, etc.

Following on the foregoing, the compositions containing the cytokine modulating agents can be formulated for immediate release or for delayed or controlled release. In this latter regard, certain embodiments, e.g., an orally administered product, can be adapted for controlled release. Controlled delivery technologies can improve the absorption of the cytokine modulating agent in a particular region, or regions, of the gastrointestinal tract in the case of orally administered doses or in the respiratory tract in the case of nasal or inhalation administered doses. Controlled delivery systems are designed to deliver the cytokine modulating agent in such a way that its level is maintained within a therapeutically effective window whereby effective and safe blood levels are maintained for a period as long as the delivery system continues to deliver the cytokine modulating agent with a particular release profile. Controlled delivery of orally administered the cytokine modulating agents typically and preferably produce substantially constant blood levels of the active over a period of time as compared to fluctuations observed with immediate release dosage forms. Controlled delivery of inhalation administered the cytokine modulating agents typically and preferably produce substantially constant levels of the active in the tissue of the respiratory tract over a period of time as compared to fluctuations observed with immediate release dosage forms. For some the cytokine modulating agents, maintaining a constant blood and/or tissue concentration of the cytokine modulating agent throughout the course of treatment is the most desirable mode of treatment as immediate release of the cytokine modulating agent may cause the blood or tissue level of the active to peak above that level required to elicit the most desired response. This results in waste of the cytokine modulating agent and/or may cause or exacerbate toxic side effects. In contrast, the controlled delivery of the cytokine modulating agent can result in optimum therapy; not only reducing the frequency of dosing, but also reducing the severity of side effects. Examples of controlled release dosage forms include dissolution-controlled systems, diffusion-controlled systems, ion exchange resins, osmotically controlled systems, erodible matrix systems, pH independent formulations, and gastric retention systems.

The pharmaceutical compositions provided by the present disclosure can be formulated in a unit dosage form. A unit dosage form refers to a physically discrete unit suitable as a unitary dose for patients undergoing treatment, with each unit containing a predetermined quantity of the cytokine modulating agent calculated to produce the desired response, especially a reduction in IL-6 and/or IL-8 and/or an increase in IL-12, IFN-alpha and/or IFN-gamma. A unit dosage form can be for a single daily dose, for administration 2 times per day, or one of multiple daily doses, e.g., 3 or more times per day. When multiple daily doses are used, a unit dosage form can be the same or different for each dose. One or more dosage forms typically comprise a dose, which can be administered to a patient at a single point in time or over a time interval, e.g., administered intravenously.

Of course, one may vary the amount and/or frequency of the dosing with time as symptoms of acute respiratory distress worsen or subside and/or manifestation of normal immune response appear, as appropriate. For example, one may monitor the level of IL-6 and or IL-8 in a patient and adjust the dosage, its frequency, etc. to either drop their levels to normal levels or to a more controlled, moderate level sufficient to maintain an immune response to the pathogen while preventing hyperinflammation. Similarly, it may be desirable to administer a large initial dose to essentially shock the system to prevent or address hyperinflammation and/or a cytokine storm followed by a lower dose to maintain the immune response, but guard against subsequent hyperinflammation and/or another cytokine storm. Furthermore, one may increase the dose or issue a large dose if the patients symptoms worsen after treatment has begun.

Similarly, in the case of addressing those triggers or pathogens that have a low or poor induction of the immune response, administration of the cytokine modulating agent may only be necessary to affect a sufficient initiation or enhancement of the immune response. For example, one may monitor the level of IL-12, IFN-alpha and/or IFN-gamma to see whether and/or when a normal level or levels sufficient to maintain an immune response to the trigger, pathogen or the like are attained and discontinue the administration. On the other hand, if the immune system of the individual is compromised or insufficiently active, it may be necessary to continue the administration of the cytokine modulating agent to maintain a sufficient immune response throughout the course of treatment.

As noted, the cytokine modulating agent(s), more appropriately, the pharmaceutical compositions comprising the cytokine modulating agent(s), can be administered through any conventional method. The specific mode of application or administration is, in part, dependent upon the form of the pharmaceutical composition, the primary purpose or target of its application (e.g., the application may be oral if intending to address the disease generally or by nasal application or inhalation if intending to address primarily the symptom of acute respiratory distress syndrome. Suitable modes of administration include, for example, intradermal, intramuscular, intraperitoneal, intravenous, subcutaneous, intranasal, epidural, oral, sublingual, intracerebral, intravaginal, transdermal, rectal, nasal or inhalation. The preferred modes of administration, particularly where one is addressing respiratory distress syndrome, are oral, intravenous infusion, nasal application, or inhalation. The former allows for absorption through epithelial or mucous linings of the gastrointestinal tract (e.g., oral mucosa, rectal, and intestinal mucosa, etc.) while the latter allows direct application to the tissue of the respiratory tract that is manifesting the symptoms of respiratory distress: intravenous infusion addresses the condition more systemically. In following, again, depending in part upon the form of the administration and the purpose of the administration, the pharmaceutical compositions of the present disclosure can be administered systemically and/or locally. Finally, the form of the pharmaceutical composition containing the cytokine modulating agents and its delivery system varies depending upon the parameters already noted. For example, orally administered pharmaceutical compositions of the present teaching can be in encapsulated form, e.g., encapsulated in liposomes, or as microparticles, microcapsules, capsules, etc.

Again as noted above, the cytokine modulating agents can be used as is, i.e., as 100% of the composition to be applied; however, the cytokine modulating agents are preferably incorporated into a pharmaceutical composition in which the cytokine modulating agent(s) account for from about 0.01 to about 99 weight percent of the pharmaceutical composition. Preferably, the cytokine modulating agent(s) will comprise from about 0.5 to about 30 wt. %, more preferably from about 0.5 to about 20 wt. %, most preferably from about 1.0 to about 10 wt. % of the pharmaceutical composition. Another factor playing into the concentration of the cytokine modulating agent in the pharmaceutical composition is the dose or rate of application of the compounds to the patient, as noted above. As noted previously, dosing itself depends upon a number of factors including the concentration and/or purity of the cytokine modulating agent(s), the efficacy thereof, the individual to whom the pharmaceutical is to be administered, the mode of administration, the form in which the pharmaceutical composition is to be administered, the disease or symptom to be addressed, etc. Generally speaking, an appropriate dose of the cytokine modulating agent, or of the pharmaceutical composition comprising the cytokine modulating agent, can be determined according to any one of several well-established protocols including in-vitro and/or in-vivo assays and/or model studies as well as clinical trials. For example, animal studies involving mice, rats, dogs, and/or monkeys can be used to determine an appropriate dose of a pharmaceutical compound. Results from animal studies are typically extrapolated to determine appropriate doses for use in other species, such as for example, humans.

The foregoing factors as well as the application thereof in formulating the pharmaceutical compositions to be administered in the present teaching are well understood and appreciated in the art whereby the final or actual concentration in the pharmaceutical composition and/or the dose can readily be determined based up simple dose-response testing and the like. For example, an appropriate oral dosage for a particular pharmaceutical composition containing one or more cytokine modulating agents will depend, at least in part, on the gastrointestinal absorption properties of the compound, the stability of the compound in the gastrointestinal tract, the pharmacokinetics of the compound and the intended therapeutic profile.

An appropriate controlled release oral dosage and ultimate form of a pharmaceutical composition containing the cytokine modulating agents will also depend upon a number of factors. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Again, it is to be expected that certain compounds are absorbed primarily from the small intestine whereas others are absorbed primarily through the large intestine. It is also to be appreciated that while it is generally accepted that compounds traverse the length of the small intestine in about 3 to 5 hours, there are compounds that are not easily absorbed by the small intestine or that do not dissolve readily. Thus, in these instances, the window for active agent absorption in the small intestine may be too short to provide a desired therapeutic effect in which case large intestinal absorption must be channeled and/or alternate routes of administration pursued.

When additional pharmacological actives are also present in the compositions according to the present teaching, the amount by which they are present and/or the dosage amount will typically be consistent with their conventional concentration and rates of application. For example, such other actives will be present in an amount of from about 0.5 to about 30 wt. %, more preferably from about 0.5 to about 20 wt. %, most preferably from about 1.0 to about 10 wt. % of the pharmaceutical composition. Of course, as noted, the combination of these other pharmacological actives with a cytokine modulating agent also provide enhanced performance and/or synergy whereby the amounts of each and/or the dose of each is generally less than required for the use of the individual active compounds on their own.

Without further elaboration, it is believed that one skilled in the art can, using, the preceding description, utilize the present invention to its fullest extent. The following preferred specific embodiments are, therefore, to be construed as merely illustrative, and not limitative of the remainder of the disclosure in any way whatsoever.

### EXAMPLES

### Example 1—IL6 and IL8 Inhibition

Cytokine inhibitory studies were performed to demonstrate the efficacy of Terminalia chebula fruit extract (TC) in downregulating interleukin 6 and interleukin 8. Identity of TC was determined by HPLC analysis which identified 12 major peaks corresponding to constituents with>2% concentration. The 12 constituents together accounted for 74% of the total extract composition, with approximately 50% of the total HPLC area accounted for by the top 4 peaks. The two largest peaks corresponded to the tannins chebulinic acid (22.2%) and chebulagic acid (17.4%). A third minor peak corresponded to gallic acid (4.5%). These identifications were confirmed by comparison to a reference chromatogram obtained from a pure mixture of the three metabolites.

The general methodology followed for this study was as follows:

Human Keratinocyte Cell Culture: Human adult epidermal keratinocytes were grown using EpiLife Media (60 μM calcium) supplemented with 0.2% v/v bovine pituitary extract, 1 μg/ml recombinant human insulin-like growth factor-I, 0.18 82 g/ml hydrocortisone, 5 μg/ml bovine transferrin, 0.2 ng/ml human epidermal growth factor. The cells were cultured at 37 C and 5% CO2. When a sufficient number of cells had been grown, they were seeded into 24-well plates and grown until confluent. Once confluent the cells were cultured overnight in hydrocortisone free EpiLife Media.

Preparation of Urban Dust & treatment: Urban dust was obtained from Sigma Chemicals (Standard Reference Material 1649b) and prepared at 10 mg/ml in hydrocortisone free EpiLife media. The urban dust solution was then sonicated for 10 minutes on ice. The test material was prepared in hydrocortisone free media supplemented with 100 ug/ml urban dust. Dexamethasone (39 ug/ml, 100 uM) was used as a positive control, while cells treated with urban dust alone were used as an untreated control. Finally, one set of cells was not exposed to urban dust and was used to establish a baseline for each cytokine. After the treatments were applied the cells were incubated for 24 hours as described above. At the end of the incubation period the cell culture media was collected to assay for cytokine release.

A series of standards was prepared and 100 μl of each of these standards was dispensed into two wells (duplicates) in the ELISA well plate. Subsequently, 100 μl of each cell culture media sample was added to additional wells and the plate was incubated for two hours at room temperature. After the incubation the plate was washed three times as described above. Once the last wash was removed, 100 μl of a biotin conjugated detection antibody was added. After incubating the plate for two hours at room temperature the plate was washed again as described above. 100 μl of HRP-streptavidin was then added to each well and the plate was incubated for 20 minutes at room temperature. Once the last wash was removed, 100 μl of substrate solution (hydrogen peroxide+tetramethylbenzidine as a chromogen) was added to each well. Once a sufficient level of color development had occurred, 50 μl of stop solution 22N sulfuric acid) was added to each well and the plate was read at 460 nm.

The results of the study are presented in Tables 1 and 2. As indicated, Terminalia chebula fruit extract (TC) manifested a marked down regulation/expression of IL-6 and IL-8. With respect to the downregulation of IL-6 the Terminalia chebula fruit extract performed markedly better than the positive control, dexamethasone, a known IL-6 inhibitor, particularly on a comparable weight basis. With respect to IL-8, Terminalia chebula fruit extract performed markedly better regardless of the weight.

TABLE 1
Inhibition of cytokine IL-6

| Treatment | | IL-6 (pg/ml) | *p = <0.05 |
|---|---|---|---|
| No IL-6 stimulator/ | | 22.4 ± 3.4 | |
| Untreated cells | | | |
| Treated with IL-6 stimulator | | 251.1 ± 23.0 | * Significantly different |
| | | | from Untreated |
| 100 | μM Dexamethasone | 61.0 ± 13.9 | * Significantly different |
| (39 | μg/ml) | | from Untreated |
| 20 | ug/ml TC | 29.1 ± 13.1 | * Significantly different |
| | | | from Untreated |
| 10 | μg/ml TC | 54.4 ± 9.1 | * Significantly different |
| | | | from Untreated |
| 5 | μg/ml TC | 76.3 ± 2.8 | * Significantly different |
| | | | from Untreated |
| 2.5 | μg/ml TC | 95.7 ± 5.2 | * Significantly different |
| | | | from Untreated |

TABLE 2
Inhibition of cytokine IL-8

| Treatment | | IL-8 (pg/ml) | *p = <0.05 |
|---|---|---|---|
| No-IL-8 stimulator/ | | 46.8 ± 9.3 | |
| Untreated cells | | | |
| Treated with IL-8 stimulator | | 302.1 ± 17.6 | * significantly different |
| | | | from Untreated |
| 100 | μM Dexamethasone | 86.9 ± 24.5 | * significantly different |
| (39 | μM/ml) | | from Untreated |
| 20 | μg/ml TC | 62.5 ± 11.0 | * significantly different |
| | | | from Untreated |
| 10 | μg/ml TC | 79.5 ± 7.8 | * significantly different |
| | | | from Untreated |
| 5 | μg/ml TC | 110.2 ± 1.3 | * significantly different |
| | | | from Untreated |
| 2.5 | μg/ml TC | 132.5 ± 11.6 | * significantly different |
| | | | from Untreated |

### Example 2—Human Bronchial Epithelial Cells

Having demonstrated the effect of the cytokine modulating agents on IL-6 and IL-8 in a gene assay, a further experiment was performed to demonstrate the efficacy on human bronchial epithelial cells (HBEpC). HBEpC provide an excellent model for all aspects of respiratory epithelial function and diseases, particularly those related to airway viral/bacterial infections, including tissue repair mechanisms, signaling changes and potential treatments relevant to lung injuries, mechanical and oxidative stress, inflammation, etc., including that arising from or associated with pulmonary diseases and smoking. In this study, the inflammatory cytokines IL-6 and IL-8 were induced through pretreatment of the HBEpC with lipopolysaccharide (Cat #L2630, Sigma, Saint Louis, Mo.). After 3 hours of treatment the test and control materials were added to the cultures which were then incubated at room temperature for 48 hrs. Thereafter the IL-8 and IL-6 levels of the test cultures were measured by ELISA.

The results of the HBEpC study are presented in Tables 3 and 4. Each experimental condition and control were assayed at least in six replicates and statistical significance assessed with paired Student test. In conducting the assay, total insoluble proteins were quantified to determine the effect of the test materials on cell proliferation according to the method described by Voigt (Voigt, W. Sulforhodamine B assay and chemosensitivity. Methods Mol Med. 2005; 110:39-48). As evident from the results presented in Tables 3 and 4, Terminalia chebula extract (TC) was found to have a statistically significant inhibitory effect on the secretion of IL-8 at all levels tested and on IL-6 at the higher levels in HBEpC whereas, surprisingly, Dexamethasone (positive control) did not show any positive results.

TABLE 3
Inhibition of cytokine Il-6

| Treatment | | IL-6 (% control) | *p = <0.05 |
|---|---|---|---|
| Treated with IL-6 | | 100 | z |
| stimulator (control) | | | |
| 20 | μM Dexamethasone | 77 | Not significant |
| 10 | μg/ml TC | 60 | * significantly different |
| | | | from control |
| 5 | μg/ml TC | 88 | Not significant |
| 2.5 | μg/ml TC | 93 | Not significant |

TABLE 4
Inhibition of cytokine IL-8

| Treatment | | IL-8 (% control) | *p = <0.05 |
|---|---|---|---|
| Treated with IL-8 | | 100 | z |
| stimulator (control) | | | |
| 20 | μM Dexamethasone | 98 | Not significant |
| 40 | μM Dexamethasone | 91 | Not significant |
| 10 | μg/ml TC | 72 | * significantly different |
| | | | from control |
| 5 | μg/ml TC | 61 | * significantly different |
| | | | from control |
| 2.5 | μg/ml TC | 56 | * significantly different |
| | | | from control |

### Example 3—Ciliated Airway Tissue

To further demonstrate the efficacy of the cytokine modulating agents in humans, another in-vitro study was conducted employing 3D ciliated airway tissues which model and represent a highly physiological, three-dimensional cellular system of Human Bronchial Epithelial Cells (HBEpC). The test materials were stored at room temperature and stock solutions were prepared in DMSO (Dexamethasone) or sterile distilled water (Terminalia Chebula fruit extract) at 20 mg/ml. Further dilutions were made in sterile distilled water (dH2O). Samples were added in at least three replicates to normal, non-diseased human bronchial epithelial cells (HBEpC cat. #502K-05a, Cell Applications, San Diego) cultured in Bronchial/Tracheal Epithelial Cell Growth Medium (cat. #511-500, lot #35703, Cell Applications). After three hours of incubation with test materials, cells were exposed to endotoxin—1 μg/ml LPS (lipopolysaccharides from Escherichia coli O113:H10, Associates of Cape Cod, East Falmouth, Mass.) and the incubation continued for an additional 48 hours.

The tissue cultures were then evaluated for IL-6 which was quantified by Sandwich Elisa. All colorimetric measurements were performed using Molecular Devices microplate reader MAX190 and SoftMax3.1.2PRO software. Statistical significance was assessed with paired Student test. Deviations of ≥20% as compared to water control with p values below 0.05 were considered statistically significant.

The results of the 3D ciliated airway tissue study are presented in Table 5. As with Example 3, each tissue culture was standardized individually and each test sample and control was assayed at least in six replicates. As indicated both Terminalia chebula fruit extract (TC) and Emblica officinalis fruit extract (EMB) as well as the combination of the two manifested a reduction in IL-6: TC again showing a particularly marked reduction.

TABLE 5
Inhibition of cytokine IL-6

| Treatment | IL-6 (% control) |
|---|---|
| Treated with IL-6 | 100 |
| stimulator (control) | |

| | | |
|---|---|---|
| 100 | μg/ml Dexamethasone | 123 |
| 100 | μg/ml TC | 47 |
| 50 | μg/ml TC | 64 |
| 100 | μg/ml EMB | 89 |
| 50 | μg/ml EMB | 84 |
| 50:50 | μg/ml TC + EMB | 73 |
| 25:25 | μg/ml TC + EMB | 75 |

### Example 4—Interferon

Having demonstrated the efficacy of select cytokine modulating agents in down regulating interleukins associated with inflammation, especially those associated with respiratory distress syndrome, e.g., hyperinflammation and cytokine storm, a series of experiments was conducted to demonstrate their ability in inducing and/or enhancing the innate immune response, namely the interferon response. Specifically, the ability of Terminalia chebula fruit extract (TC), Emblica officinalis fruit extract (EMB) and combinations thereof to boost the innate and adaptive immunity through upregulation of interferon alpha and interferon gamma, respectively, was evaluated. In this study, Dexamethasone (DEX) was selected as the positive control to demonstrate the efficacy of the present inventive composition in enhancing the interferon response to lipopolysaccharide (LPS) or viral proteins in Human bronchial epithelial cells 3D model system.

In this study, 3D ciliated airway tissues (cat. #502-3D-24) were obtained from Cell Applications (San Diego, Calif.) and were cultured in maintenance medium (cat. #511M-3D-100, Cell Applications). The respective test materials were stored at room temperature and samples were solubilized in sterile water (LPS, TC, EMB) or DMSO (DEX) at 20 mg/ml, afterward all further dilutions were made in sterile distilled water. All test materials except LPS were assayed at 100 μg ml (LPS was added topically at 1 μg/ml on Day 2). Furthermore, TC and EMB were tested at 50 μg/ml and in two combinations: 50 μg/ml:50 μg/ml and 25 μg/ml:25 μg/ml. Samples were added to the feeder chamber medium contacting the basal side of the tissues on Day 1, then also topically on Day 2, together with LPS. All experimental conditions were tested in triplicates.

Twenty-four hours after pre-incubation with the test substances, the tissue cultures were exposed to 1 μg/ml endotoxin LPS and the incubation was continued for 48 additional hours. Following the incubation, the experiment tissue culture-conditioned medium was stored at −20° C. until further processing. Subsequently, IFN-α and IFN-γ were quantified in the tissue culture-conditioned medium by sandwich ELISA. All colorimetric measurements were performed using Molecular Devices microplate reader MAX190 and SoftMax3.1.2PRO software.

TABLE 6
Upregulation of Interferon alpha and Interferon gamma

| | | Interferon alpha | Interferon alpha |
|---|---|---|---|
| Treatment | | (pg/ml) | (pg/ml) |
| Water (control) | | 6 | 9 |
| 100 | μg/ml Dexamethasone | 6 | 20 |
| 100 | μg/ml TC | 10 | 39 |
| 50 | μg/ml TC | 5 | 22 |
| 100 | μg/ml EMB | 6 | 21 |
| 50 | μg/ml EMB | 5 | 16 |
| 50:50 | μg/ml TC + EMB | 10 | 22 |
| 25:25 | μg/ml TC + EMB | 6 | 20 |

The results of the study are presented in Table 6. As shown, there was an extremely low level of both interferons in the control (water-treated) medium and while the positive control (dexamethasone) failed to induce an interferon response with respect to interferon alpha, it did induce the production of interferon gamma. On other hand, IFN-alpha was significantly upregulated by the higher concentration of TC (100 μg/ml) and the higher combination of TC and EMB, but failed to show a significant change at the lower levels. Although interferon alpha is certainly important, the more important of the interferons is the interferon gamma, which, as shown, was markedly induced or enhanced by the cytokine modulating agents.

Given the results with interferon gamma and in light of the fact that interleukin 12 is known to induce interferon gamma, it is expected that these materials will result in an upregulation and high levels of IL-12.

### Example 5: Capsule Formulation

Capsule compositions were prepared for use in conducting a blind test to demonstrate the in-vivo efficacy of Terminalia chebula fruit extract (TC) in the method of the present teaching. The formulation of the test composition was as presented in Table 7: a placebo was also prepared using the same ingredients less the TC. All the excipients used in the capsules are either USP or NG grade. Hard gelatin capsules with TC and placebo were manufactured. In order to maintain the uniformity and consistency, a quantity of the placebo blend was prepared sufficient for the capsules with TC and the placebo capsules. The placebo blend was split to produce 6,000 active capsules containing TC and 4,020 placebo capsules. After manufacturing all the capsules were filled in 3 oz white HDPE round bottle, a silica absorbent pouch was added to the bottles.

TABLE 7
Capsule Formulation Composition

| | Ingredient | Amount in mg |
|---|---|---|
| 1 | Terminalia chebula fruit extract (TC) | 250 |
| 2 | Avicel pH 101 (microcrystalline cellulose) | 20 |
| 3 | Starch 1500 | 17.5 |
| 4 | Stearic acid NF (powder) | 2 |
| 5 | Cab-O-Sil 0.5 | 0.5 |

### Example 5: Tablet Formulation

Tablets for use in the method of the present teaching are prepared using the formulation set forth in Table 8 as follows: Terminalia chebula fruit extract is granulated with starch paste to make a free-flowing powder. All ingredients but for the stearic acid NF are blended for 25 min. in a blender. The stearic acid NF is the screened and into the formulation and the combination blended for an additional 5 min. The final composition is then compressed into tablets using a 7/16-in standard concave tooling.

TABLE 8
Tablet Formulation Composition

| | Ingredient | Amount in mg |
|---|---|---|
| 1 | Terminalia chebula fruit extract (TC) | 500 |
| 2 | Avicel pH 101 (microcrystalline cellulose) | 84.0 |
| 3 | Starch 1500 | 75.0 |
| 4 | Stearic add NF (powder) | 8.5 |
| 5 | Cab-O-Sil 0.5 | 2 |

Without further elaboration, it is believed that one skilled in the art, using the preceding description, can utilize the present invention to its fullest extent. Furthermore, while the present invention has been described with respect to aforementioned specific embodiments and examples, it should be appreciated that other embodiments, changes and modifications utilizing the concept of the present invention are possible, and within the skill of one in the art, without departing from the spirit and scope of the invention, The preceding preferred specific embodiments are, therefore, to be construed as merely illustrative, and not limitative of the remainder of the disclosure in any way whatsoever.

1. A method of preventing, inhibiting, mitigating and/or treating acute respiratory distress syndrome associated with or caused by the SARS-Cov-2 virus and/or the disease Covid-19 comprising administering an effective amount of a cytokine modulating agent selected from the group consisting of Terminalia chebula fruit extract, Emblica officinalis fruit extract. Terminalia bellerica fruit extract and Terminalia arjuna extract to an individual who has contracted Covid 19.

2-7. (canceled)

8. The method of claim 1 wherein the cytokine modulating agent is administered to an individual manifesting a symptom of respiratory distress.

9. The method of claim 8 wherein the symptom is hyperinflammation.

10. The method of claim 1 wherein the cytokine modulating agent downregulates IL-6 and/or IL-8 and/or their corresponding downstream genes or the production thereof.

11-13. (canceled)

14. The method of claim 1 wherein the cytokine modulating agent is selected from the group consisting of tannin rich Terminalia chebula fruit extract, tannin rich Emblica officinalis fruit extract and tannin rich Terminalia bellerica fruit extract.

15. The method of claim 14 wherein the tannin rich extract has at least 30% by weight tannins.

16-25. (canceled)

26. The method of claim 1 wherein the cytokine modulating agent is Terminalia chebula fruit extract.

27. The method of claim 1 wherein the cytokine modulating agent is Emblica officinalis fruit extract.

28. The method of claim 1 wherein the cytokine modulating agent is Terminalia bellerica fruit extract.

29. The method of claim 1 wherein the cytokine modulating agent is Terminalia arjuna extract.

30. The method of claim 1 wherein the cytokine modulating agent is tannin rich Terminalia chebula fruit extract.

31. The method of claim 1 wherein the cytokine modulating agent is tannin rich Emblica officinalis fruit extract.

32. The method of claim 1 wherein the cytokine modulating agent is tannin rich Terminalia bellerica fruit extract.

33. The method of claim 8 wherein the symptom is a cytokine storm.

34. The method of claim 1 wherein the amount and/or frequency at which the individual is administered the cytokine modulating agent is adjusted based upon the manifestation of respiratory distress.

35. The method of claim 1 wherein the amount and/or frequency at which the individual is administered the cytokine modulating agent is adjusted based upon the individual's level of IL-6 and/or IL-8.

36. The method of claim 1 wherein the administration of the cytokine modulating agent is maintained until the individual's level of IL-12, IFN-alpha and/or IFN-gamma return to normal levels or a level sufficient to maintain an immune response to SARS-CoV-2.

37. The method of claim 14 wherein the tannin rich extract has at least 40% by weight tannins.

38. The method of claim 14 wherein the tannin rich extract has at least 20% by weight tannins.

39. The method of claim 1 wherein the cytokine modulating agent is administered intravenously, nasally, or through inhalation.
